(19) United States
(12) Patent Application Publication
(10) Pub. No.: US 2005/0276865 A1
(43) Pub. Date: Dec. 15, 2005

(54) Peroxide compounds for the prevention and treatment of sexual dysfunction in humans

(76) Inventors: Servet Buyuktimkin, Robbinsville, NJ (US); Nadir Buyuktimkin, Robbinsville, NJ (US); James L. Yeager, Lake Forest, IL (US)

Correspondence Address: WELSH & KATZ, LTD, 120 S RIVERSIDE PLAZA, 22ND FLOOR, CHICAGO, IL, 60606, US

(21) Appl. No.: 11/131,520

(22) Filed: May 18, 2005

Related U.S. Application Data
(60) Provisional application No. 60/572,904, filed on May 20, 2004.

(51) Int. Cl. A61K 31/19; A61K 33/40

(52) U.S. Cl. 424/616; 514/568

(57) ABSTRACT

A composition and method for treatment or prevention of human sexual dysfunction are disclosed. The method contemplates the topical application to a genital organ of composition containing an effective sexual arousal-inducing amount of a peroxide compound. Preferred peroxide compounds include benzoyl peroxide and carbamide peroxide, and mixtures thereof.

### CROSS-REFERENCE TO RELATED APPLICATION

This application claims the benefit of the filing date of U.S. Application Ser. No. 60/572,904, filed May 20, 2004.

### TECHNICAL FIELD

The present invention is related to a composition and method for long- or short-term treatment of sexual dysfunction in humans by a topical application using one or more peroxide compounds. A contemplated peroxide can be used alone or in a suitable formulation or matrix. A contemplated composition can be administered by the use of a dispenser or applicator for delivery to the desired anatomical location.

### BACKGROUND OF THE INVENTION

Male Sexual Dysfunction

Sexual dysfunction in male, commonly called male erectile dysfunction (ED) is clinically defined as the inability to attain and maintain an erection of the penis sufficient to permit satisfactory sexual intercourse [NIH Consensus Conference, “Impotence” J. Amer. Med. Assoc. 270:83-90 (1993)]. This disorder affects 10 to 30 million men in the US, more than 140 million men worldwide, and has been reviewed in the literature [Furlow, “Prevalence of Impotence in the United States”, Med. Aspects Hum. Sex, 19:13-16 (1985); Kaiser, “Erectile dysfunction in the aging man”, Medical Clinics North America, 83:1267-1278 (1999)].

The causes of ED can be psychogenic, hormonal or vasculogenic; the most common causes are vascular diseases in older men and psychogenic disorders in younger men [Morely et al., “Impotence: The Internist's Approach to Diagnosis and Treatment”, Adv. Intern Med. 38:151-168 (1993)]. Urologic, neuropathic, and endocrine causes are less common. The etiology of ED is diverse and can be vascular (arteriosclerotic, venous leakage, arteriovenous malformations, local trauma), neuropathic (stroke, multiple sclerosis, temporal lobe epilepsy, spinal cord trauma, spinal cord tumor, autonomic neuropathy, sensory neuropathy, psychological depression, performance anxiety, stress), or endocrine (diabetes mellitus, hypogonadism, hyperprolactinemia, hypothyroidism, Cushing's syndrome) in origin [Kaiser, “Erectile dysfunction in the aging man”, Medical Clinics North America, 83:1267-1278, 1999]. In addition, some medications have been shown to cause ED (Kaiser, “Erectile dysfunction in the aging man”, Medical Clinics North America, 83:1267-1278 (1999)]. Less frequent causes include systemic (renal failure, chronic obstructive pulmonary disease, cirrhosis, leprosy, myotonia dystrophica), and nutritional (obesity, protein malnutrition, zinc deficiency) disorders [Morely et al., “Impotence: The Internist's Approach to Diagnosis and Treatment”, Adv. Intern Med. 38:151-168 (1993)].

As a man ages, the blood supply to the penis can decrease due to the increased amounts of fat that are deposited in the penile arteries. Consequently, the muscle fibers inside the penis, whose function is to regulate blood flowing through the blood vessels, begin to transform into connective tissue. Oxygen in the blood suppresses transforming growth factor (TGF-β1), an immune system cytokine that is produced by smooth muscle cells. TGF-β1 appears to stimulate collagen production in the corpus cavernosum, which can lead to erectile dysfunction. The greater the decrease of oxygen and therefore the increase of TGF-β1, the greater is the amount of collagen accumulated in the connective tissue [Nehra et al., “Transforming Growth Factor-Beta 1 (TGF Beta 1) is sufficient to induce Fibrosis of Rabbit Corpus Cavernosum”, J. Urol. 162:910-915 (1999)]. The result of increasing amounts of collagen is an increased difficulty in getting an erection.

The penis is composed of three bodies of erectile tissue; a pair of parallel spongy columns called the corpora cavernosa and a central chamber called corpus spongiosum, which surrounds the urethra and terminates in-the glans penis. The lateral expansion of the urethra in the glans penis is called fossa navicularis. Because collagen is the major component in the structural tissues it also constitutes the main component of elastic fibrous structure of erectile tissues of penis. The corpora cavernosa are encompassed by a thick collagenous band called the tunica albugenia. In the non-erect state or flaccid state, vasoconstriction of multiple cork-screw shaped blood vessels called the helicine arterioles that branch off the deep penile cavernosal artery and terminate in the lacunar spaces, causes a large pressure gradient between the deep penile cavernosal artery and the lacunar spaces, and thus maintains the flaccid state of the penis.

The physiology of penile erection involves interaction of the vascular, neurological, and hormonal systems of the body. During erection, the penis acts as a fluid-filled capacitor, accumulating blood under pressure. Thus, erection is produced by vasodilation of two arteries entering the paired corpora cavernosa (parallel vascularized cylinders extending from the ischial tuberosity to the glans [Church, The Medical Forum. Impotence, Part 2: Treatment, Harvard Medical School Health Letter, October, 1989]. Subsequent relaxation of the trabecular smooth muscle results in the expansion of the lacunar spaces and subsequent filling of these spaces with blood, followed by the trapping of this blood due to the elongation and compression of the draining veins, and activating the endothelial nitric oxide synthase.

The key mediator of smooth muscle relaxation is nitric oxide (NO), which acts by increasing the cellular level of 3′,5′-cylic guanosine monophosphate (cGMP) [Maggi et al., “Erectile Dysfunction: From Biochemical Pharmacology to Advances in Medical Therapy”, European J. Endocrinology, 143:143-154 (2000)]. Upon stimulation, nitric oxide is released from non-adrenergic, noncholinergic nerve terminals and diffuses into the underlying smooth muscle in the helicine resistance arterioles as well as the trabecular smooth muscle of the corpus cavernosum. This endothelial-derived NO diffuses into the smooth muscle, further enhancing relaxation.

In addition, nitric oxide binds to the heme of soluble guanylate cyclase in the smooth muscle cells, stimulating the synthesis of cGMP. As a result of increased CGMP synthesis, intracellular calcium levels decrease, and smooth muscle relaxation ensues. Other vasodilators such as vasoactive intestinal peptide and calcitonin gene-related peptide can also be released from nerve terminals and trigger smooth muscle relaxation via specific receptors and increasing intracellular cyclic adenosine monophosphate (cAMP) synthesis.

Oxygen also enhances the production of prostaglandin E₁ (alprostadil), an agent of choice to enhance erection, from the muscle. Prostaglandin E₁ is synthesized by the smooth muscle cells and can further enhance smooth muscle relaxation by binding to specific PGE (EP2 and/or EP4) receptors on the surface of the smooth muscle cells and elevating cAMP synthesis. During erection, the trabecular smooth muscle relaxes and permits an increased inflow of arterial blood. As the corpora cavernosum expands outward and fills with blood, the draining venules are pressed against the tunica albugenia and occluded. The blockage of outflow results in veno-occlusion and the corporal pressure rises to about 100 mm Hg, and erection follows.

Despite the arterial blood supply via the helicine resistance arterioles, at flaccidity, the penile blood oxygen pressure is about 25-40 mm Hg and the corpus cavernosum trabecular smooth muscle is contracted. It is thought these changes in oxygen tension play an active role in regulating penile erection. At low oxygen tensions, measured in the flaccid state of the penis, the synthesis of nitric oxide is inhibited, preventing trabecular smooth muscle relaxation. Following vasodilatation of the resistance arteries, the increase in arterial flow raises oxygen tension. In the oxygen-enhanced environment, autonomic dilator nerves and the endothelium are able to synthesize nitric oxide, mediating trabecular smooth muscle relaxation.

Oxygen tension can regulate the types of vasoactive substances present in this vascular bed; i.e., at low oxygen tensions, vasoconstrictors (such as norepinephrine and endothelin) can predominate, whereas at high oxygen tension, vasodilators such as nitric oxide as well as prostaglandins are produced in situ. It is also postulated that the difference in oxygen tension in the flaccid and erect states results in the regulation of synthesis of cytokines, autacoids, growth and vasoactive factors, which play a major role not only in trabecular smooth muscle tone but in connective tissue metabolism as well. Thus, in flaccidity, reduced oxygen tension leads to trabecular connective tissue synthesis, and during erection, increased oxygen tension leads to reduced connective tissue synthesis and collagen degradation [Nehra, “Oxygen Levels and Their Effects on Erectile Function”, Family Urology, 19-24 (1997)].

Erectile function is a coordinated interaction of the nervous system, blood supply, and hormonal activity. An increase of arterial inflow of blood to the penis and a concomitant decrease of venous outflow produces the erection [Morely et al., “Impotence: The Internist's Approach to Diagnosis and Treatment”, Adv. Intern Med. 38:151-168 (1993); Krane et al., “Impotence”, N. Engl. J. Med. 321:1648-1659 (1989)]. A network of tiny distensible veins, known as sinusoids, swells from the temporary increase in blood flow, and causes the penis to enlarge and stiffen. The expanded sinusoids compress veins that normally drain blood from the organ, and thus, trap blood within the sinusoidal network. The restricted blood outflow leads to an increase in intracavernosal pressure to a value that approximates the mean systemic arterial blood pressure. Impairment of the mechanisms that relax corporal smooth muscle, penile arteries, or sinusoids can result in varying degrees of ED [Owen et al., “Topical Nitroglycerin: A Potential Treatment for Impotence”, J. Urol. 141: 546 (1989)].

Erections can be important in modulating trabecular connective tissue metabolism and to maintain a functional connective tissue/smooth muscle ratio crucial for erectile function. This balance of factors that regulate erection can also affect corpus cavernosum structure. Prolonged deoxygenation of the corpora that occur during erection can accelerate the processes leading to corporal fibrosis and failure of venoocclusion [Park et al., “The role of Oxygen Tension in Penile Erection and its Relationship to Erectile Dysfunction”, J. Digital Urol., 2000].

The important oxygenation of the penis during nighttime erections can be critical to the production of substances such as prostaglandin, which promote healthy erectile function. Evidence also suggests that in situations of poor oxygenation of the penis such as vasculogenic impotence, the reverse can be true with a double increased amount of fibrosis-producing transforming growth factors and decreased amounts of health-promoting prostaglandin E₁. This imbalance can result in a vicious cycle, which eventually results in a penis that is physiologically not capable of an erection.

Several treatments for ED have been approved and used over the course of the past several years ranging from surgery (implants) to mechanical devices (vacuum devices) to injectable vasodilators (Caverject®, Edex®) to intraurethral inserts (Muse®) to the more recent introduction of oral phosphodiesterase inhibitors such as sildenafil citrate (Viagra®), tadalafil (Cialis®) and vardenafil (Levitra®). Injectable preparations and intraurethral devices can cause local irritation problems.

Another treatment for ED being tried is injection of prostaglandin E₁ into the corpus cavernosum of the penis. Of the men who have undergone such treatment, 28 percent have achieved improved spontaneous erections and required less frequent need for the injections [Montorsi et al., “Recovery of Spontaneous Erectile Function after Nerve-sparing Radical Retropubic Prostatectomy with and without Early Intracavernous Injections of Alprostadil: Results of a Prospective Randomized Trial”, J. Urol., 158:1408-1410 (1997)].

Currently, there is no topical product specifically designed for the treatment of erectile dysfunction approved by The United States Food and Drug Administration (FDA). Considerable efforts are therefore still in progress to develop a topical product that is convenient to use, local and fast acting, and can be used on demand without a significant wait for systemic absorption. One of the topical agents of choice would be prostaglandin E₁ (alprostadil) because it is has already shown to be effective in the injectable and intraurethral format. On going efforts to cure and treat erectile dysfunction can be found in various issued U.S. patents, for example: Drizen et al. U.S. Pat. No. 6,514,536; Podolski U.S. Pat. No. 6,482,426; Buyuktimkin et al. U.S. Pat. No. 6,046,244 and No. 6,414,028 and Yeager et al. No. 6,693,135, No. 6,486,207, and No.6,323,241.

Female Sexual Arousal Disorder

Female sexual arousal disorder (FSAD) is the persistent or recurrent inability to attain, or to maintain, sufficient sexual excitement, which causes personal distress. It can be expressed as lack of subjective excitement, lack of genital response, such as lubrication and swelling, or lack of other somatic responses. Female sexual arousal disorder is one form of female sexual dysfunction, and is associated with the excitement phase.

Although increased understanding of the pathophysiology of male erectile dysfunction has progressed rapidly in the past decade and led to new therapeutic modalities, little has been done to address similar issues in women. Cardiovascular risk factors have been shown to correlate with complaints of vaginal and clitoral dysfunction. [Goldstein et al., “Gynecological factors in sexual dysfunction of the older woman”, Clin. Geriatr. Med. 7:41-61 (1991); Sadeghi-Nejad et al., “Impotence is a couple's disease: studies in female sexual dysfunction”, J. Urol. 155:677A (1996); Slob et al., “Sexuality and psychophysiological functioning in women with diabetes mellitus”, J. Sex Marital. Ther. 16:59-69 (1990)].

The correlation of cardiovascular risk factors and complaints of vaginal and clitoral dysfunction have led to suggestions that a significant degree of female sexual dysfunction is due to vascular insufficiency and therefore amenable to treatment with vasoactive agents. The underlying foundations of the normal and dysfunctional female sexual response must be considered in the context of the anatomy and physiology, summarized below. [See, generally, Goldstein and Berman, “Vasculogenic female sexual dysfunction: vaginal engorgement and clitoral erectile insufficiency syndromes”, Int. J. Impotence Res. 10: Suppl. 2, S84-S90 (1998).]

The walls of the vagina consist of three layers: (i) an inner mucosa and aglandular mucous membrane epithelium, (ii) an intermediate, highly vascularized muscularis layer, and (iii) an outer supportive fibrous mesh. The vaginal mucosa is a mucous type stratified squamous cell epithelium that undergoes hormone-related cyclical changes, such as a slight keratinization of the superficial cells during the menstrual cycle.

The arterial supply to the vagina is derived from an extensive network of branching vessels surrounding it from all sides. The anterior branch of the internal iliac artery continually bifurcates as it descends through the pelvis with a series of the newly generated vessels, each supplying the vagina to some degree. After giving off an obturator artery branch, the umbilical, and the middle rectal arteries diverge off to supply a superior and inferior vesicle artery, respectively. Between the umbilical and the mid-rectal branches there is a generation of a uterine artery, which further bifurcates to provide the vaginal artery. The internal pudendal and accessory pudendal artery also send a branch to the vaginal artery. Finally, the common clitoral artery sends a branch to the vaginal muscularis.

Nerve fibers of the vagina had previously been shown to be active in association with specific peptides which include vasoactive intestinal peptide (VIP), peptide histidine methionine (PHM), calcitonin gene related peptide (CGPP), and galanin. Genital vasodilation and subsequent increase in vaginal blood flow and lubrication have been observed upon exposure of vessels to VIP. VIP has been implicated as the neurotransmitter for mediating vaginal vasodilation and the formation of lubricating fluid during sexual arousal. Helospectin and PACAP, a potent vasodilator, belong to the same peptide family as VIP and PHM, and recent observations have been made to the effect that distributions and co-localizations of helospectin and VIP as well as PACAP and VIP have been reported in the mammalian gastrointestinal tract.

The clitoris is the homologue of the penis arising from the embryological genital tubercle. The clitoris consists of a cylindrical, erectile organ composed of three parts: the outermost glans or head, the middle corpus or body, and the innermost crura. The glans of the clitoris is visualized as it emerges from the labia minora, which bifurcate to form the upper prepuce anteriorly and the lower fronulum posteriorly. The body of the clitoris consists of two paired corpora cavernosa of about 2.5 cm in length and lacks a corpus spongiosum. The body extends under the skin at the corona to the crura. The two crura of the clitoris, formed from the separation of the most proximal portions of the corpora in the perineum, attach bilaterally to the undersurface of the symphysis pubis at the ischiopubic rami.

The main arterial supply to the clitoris is from the illo-hypogastric-pudendal arterial bed. The internal pudendal artery is the last anterior branch off the internal iliac artery. Distally, the internal pudendal artery traverses Alcock's canal, a position of the obturator fascia and lies on the inner side in apposition to the ischio-pubic ramus. In this latter location, the artery is susceptible to blunt perineal trauma. The internal pudendal artery terminates as it supplies the inferior rectal and perineal artery, which supplies the labia. The common clitoral artery continues to the clitoris. This artery bifurcates into a dorsal clitoral artery and a cavernosal clitoral artery.

The clitoris can play a major role during sexual activity in that it is not only part of what makes the sexual act enjoyable for the woman but also enhances her response to coitus upon clitoral stimulation. Clitoral stimulation can induce local autonomic and somatic reflexes causing vaginal vasocongestion, engorgement, and subsequent transudation, lubricating the introital canal making the sexual act easier, more comfortable, and more pleasurable. The more stimulation, the higher the level of arousal and the easier it is to further increase stimulations.

The female sexual response phase of arousal is not easily distinguished from the phase of desire until physiological changes begin to take place in the vagina and clitoris as well as other sexual organs. Sexual excitement and pleasure are accompanied by pelvic vasocongestion and swelling of the external genitalia including vaginal engorgement and clitoral erection.

Vaginal engorgement enables a process of plasma transudation to occur, allowing a flow through the epithelium and onto the vaginal surface. Plasma transudation results from the rising pressure in the vaginal capillary bed during the arousal state. In addition there is an increase in vaginal length and luminal diameter, especially in the distal ⅔ of the vaginal canal.

The distinction between local physiological aspects of sexual response, such as genital vasocongestion measured by vaginal photoplesmography, and subjective sexual arousal, measured by self-reporting rating scales and inventories has been clearly demonstrated in both normal and sexually dysfunctional women [Palace et al., “Differential patterns of arousal in sexually functional and dysfunctional women: Physiological and subjective components of sexual response”, Arch. Sexual Behav. 21:135-159 (1992)]. Several reliable and validated self-report inventories are recognized for measurement of female sexual function (Derogatis et al., “Psychological assessment measures of female sexual functioning in clinical trials”, Int. J. Impot. Res. 10 Suppl. 2:S 11-S116 (1998)).

Female sexual dysfunction can have its origin in abnormal arterial circulation into the vagina or clitoris during sexual stimulation, usually from atherosclerotic vascular disease can be considered a disorder of arousal. This vasculogenic female sexual dysfunction can include such clinical symptoms as delayed vaginal engorgement, diminished vaginal lubrication, pain or discomfort with intercourse, diminished vaginal sensation, diminished vaginal orgasm, diminished clitoral sensation or diminished clitoral orgasm. Traumatic injury to the ilio-hypogastric-pudendal arterial bed from pelvic fractures or blunt perineal trauma can also result in diminished vaginal/clitoral blood flow following sexual stimulation and fall into this vasculogenic category.

During the last years various attempts especially in patent literature were made to treat and prevent female sexual dysfunction. For example a method and a device for treating female sexual dysfunction using various vasodilating agents were disclosed by Place et al. (U.S. Pat. No. 5,877,216). Yeager et al. (U.S. Pat. No. 6,825,234 and No. 6,486,207) and Buyuktimkin et al. (U.S. Pat. No. 6,046,244 and No. 6,414,028) teach a method to treat female sexual disorders and formulations for the administration of prostaglandin E₁ and vasodilating agents. Garvey and Saenz de Tejada in U.S. Pat. No. 6,693,122 disclosed the utilization of nitrosated and nitrosylated potassium channel activators in the treatment and the prevention of human sexual dysfunction. Mow et al. U.S. Pat. No. 6,831,074 reported the beneficial effects of cyclic guanosine 3′,5′-monophosphate phosphodiesterase inhibitors in the curative and prophylactic treatment of mammalian sexual disorders.

Several reports from the literature indicate that oxygen is of import in female sexual arousal. Vaginal wall oxygen pressure increase during intercourse was reported by Wagner et al., “Oxygen tension of the vaginal surface during sexual stimulation in the human”, Fertil. Steril. 30:50-53 (1978). These findings were further confirmed in other mammals by Giuliano et al., “Vaginal physiological changes in a model of sexual arousal in anesthetized rats”, Am. J. Physiol. Regul. Integr. Comp. Physiol. 281:R140-R149 (2001).

Sommer et al. measured vaginal and minor labial oxygen tension of female. They reported that mean basal vaginal value was 3.8±0.9 mm Hg and mean basal pO₂ on the minor labia was 18.3±3.7 mm Hg. As soon as self-stimulation was initiated, an increase in oxygen tension occurred and continued during sexual stimulation. Just before orgasm they noted a further increase with peak values pO₂ 28.6±3.1 mmHg intravaginally and 47.3±4.1 labially [Sommer et al., “Measurement vaginal and minor labial oxygen tension for the evaluation of female sexual function”, J. Urol. 165:1181-1184 (2000)].

### BRIEF SUMMARY OF THE INVENTION

The present invention provides a new approach for the treatment or prevention of sexual dysfunction by the topical application to a genital organ of composition containing an effective sexual arousal-inducing amount of a peroxide compound. Particularly preferred peroxide compounds include carbamide peroxide (urea peroxide), benzoyl peroxide and mixtures thereof. A contemplated composition is applied on an as needed basis, and left in place. A composition can contain a peroxide compound as the only active agent, the peroxide compound can be present in combination with one or more vasodilating agents such as prostaglandin E₁ or nitric oxide precursors; i.e., organonitrates.

### DETAILED DESCRIPTION OF THE INVENTION

Currently no method is described in the literature for treatment or prevention of sexual dysfunction by the use of oxygenation of sexual tissues (genitals). It has now been found that the topical application of a peroxide compound to the genitals of a human in need thereof can be helpful to improve this disorder and thus improve sexual relationships.

More specifically, a formulation that comprises a composition containing a pharmaceutically acceptable peroxide dissolved or dispersed in a topical vehicle is particularly useful for treatment or prevention of sexual dysfunction when applied topically in an effective amount to the genitals. In practicing a contemplated method, a contemplated composition comprising an effective sexual arousal-inducing amount of a peroxide compound dissolved or dispersed in a topical vehicle is administered (applied) about fifteen minutes to about one hour prior to the time of desired effect; i.e., prior to the time at which sexual arousal is desired, and is typically left in place without removal. Preferably, the composition is applied once per day, but it can be applied more frequently such as twice or three times within a twenty-four hour period.

Typical examples of pharmaceutically acceptable peroxides are carbamide peroxide (urea peroxide) and benzoyl peroxide. The former is used as the active ingredient in over the counter (OTC) tooth whitening products at a level up to 20 percent by weight, in oral antibacterial products at about 10 weight percent, and in OTC ear wax removal products at 6.5 weight percent concentration. Benzoyl peroxide is used in the treatment of acne and is present in soaps, lotions, gels and facial masks in amounts of about 5 to about 20 weight percent. A contemplated peroxide compound can be a mixture of active ingredients such as benzoyl peroxide and carbamide peroxide.

A contemplated composition contains an amount of a peroxide compound that is effective to induce sexual arousal when applied to a genital organ. Such an amount is referred to herein as an effective sexual arousal-inducing amount of a peroxide compound, or a similar phrase. As is the case with active ingredients, such compounds can be administered at different concentrations more or less frequently to achieve a desired dosage. Typical amounts used in a given formulation are about one and about 20 percent by weight, and more preferably about 3 to about 15 weight percent, and most preferably about 5 to about 10 weight percent of the topical composition.

A contemplated composition for topical application is provided dissolved or dispersed in a topical vehicle that is preferably a semi-solid at room temperature in the form of a gel, cream or ointment. That is, the topical vehicle of the composition preferably has sufficient viscosity at room temperature that it does not pour. The topical vehicle is preferably aqueous and is readily absorbed by the skin or mucus membrane. The composition thereby provides for one or both of transdermal and transmucosal drug delivery.

The term “transdermal” drug delivery, is used in its conventional sense; i.e., to indicate delivery of a drug by passage into and through the skin and the underlying tissues and into the blood stream. The term “transmucosal” drug delivery, means delivery of a drug by passage of a drug through the mucosal and underlying tissue into the blood stream. The compositions, systems, and methods of the invention, unless explicitly stated otherwise, are to be presumed to be equally applicable to either transdermal or transmucosal modes of drug delivery.

“Penetration enhancement” or “permeation enhancement” as used herein relates to an increase in the permeability of the skin or mucosal tissue to a selected pharmacologically active agent; i.e., so that the rate at which the drug permeates through the skin or mucosal tissue is increased. “Carriers” or “vehicles” as used herein refer to carrier materials suitable for transdermal or transmucosal drug administration, and include any such materials known in the art, e.g., any liquid, gel, solvent, liquid diluent, solubilizer, or the like, which is nontoxic and does not interact with other components of the composition in a deleterious manner.

Pharmaceutically acceptable peroxides such as carbamide peroxide and benzoyl peroxide were formulated into several aqueous and non-aqueous semi-solid formulations suitable for topical pharmaceutical applications. Carbamide peroxide was found to be the preferred peroxide and has the following structure.
H₂N—CO—H₂N.H₂O₂

Carbamide peroxide, USP is a water-soluble white powder. It melts at 75-85° C. by decomposition. The compound is highly unstable in aqueous medium and is classified as oxidant. Although it is reported to be an irritant (NTF Chemical Repository), the administration of up to 20 percent of this compound in tooth bleaching products was found to be safe to soft tissues when proper procedures were followed. According to many studies, toxicity and carcinogenicity concerns appeared to be unfounded [Haywood et al., “Nightguard Vital Bleaching: How safe it is?” Quintessence Int., 22:515-523, (1991)].

Topical formulations of a peroxide compound (carbamide peroxide) were prepared in a topical vehicle that included one or more organic compounds such as in propylene glycol, glycerol, or other polyethylene glycols alone or in combination and in which the active peroxide compound was present at a most preferred level of about 5 to about 10 percent w/w, e.g., 6.5 percent w/w level. Those compositions were successfully used on normal volunteers. In addition, identical formulations were prepared either with additional vasoconstrictors or as placebo formulations without any active material. The comparative applications of these formulations to a group of healthy volunteers confirmed the erectile dysfunction alleviating properties of the peroxides utilized.

Commercial formulations of carbamide peroxide marketed for tooth whitening or ear wax removal are not suitable for use in erectile dysfunction because they a exhibited lower effect for erectile dysfunction and also were not convenient for the subject-friendly application of the drug due to their lower viscosity and unacceptable consistency for and local application and retention on the genital organs.

To improve the patient compliance and improve the activity it was preferred to use other formulations specifically designed for use with carbamide peroxide with or without vasodilating agent. An illustrative composition is shown hereinafter.

The topical application of a combination of an effective sexual arousal-inducing amount of a peroxide compound with an effective amount of a vasodilating agent has also been found useful. A vasodilating agent such as prostaglandin E₁ as is discussed hereinafter is also present in some embodiments in an amount of about 0.05 to about 1.0 percent w/w.

Illustrative vasodilating agents include organonitrates as are discussed hereinbelow, molsidomine, linsidomine chlorhydrate and S-nitroso-N-acetyl-d,l-penicillamine (“SNAP”); long and short acting α-blockers such as phenoxybenzamine, dibenamine, doxazosin, terazosin, phentolamine, tolazoline, prazosin, trimazosin, alfuzosin, tamsulosin and indoramin; ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; nimodepine, pinacidil, cyclandelate, dipyridamole and isoxsuprine; chlorpromazine; haloperidol; yohimbine; trazodone, vasoactive intestinal peptides and mixtures thereof. Prostaglandin E₁, an organonitrate and phentolamine are particularly preferred vasoactive agents for use in conjunction with the present method.

The before-mentioned Buyuktimkin et al. U.S. Pat. No. 6,046,244 and No. 6,414,028 and Yeager et al. U.S. Pat. No. 6,825,234, No. 6,693,135, No. 6,486,207, and No.6,323,241 teach illustrative compositions for topically administering prostaglandin E₁ in an effective amount for treating male and female sexual dysfunction. An illustrative prostaglandin E₁ composition is illustrated herein. The prostaglandin is present in such a formulation in a therapeutically effective amount. Therapeutic effectiveness can be assessed, in part, with increase in vaginal secretion, increase in vaginal engorgement, increase in sexual responsiveness and increase in arousal.

Organonitrate compounds are nitric oxide precursor vadodilators that can be co-administered (co-formulated) or administered separately in conjunction with a contemplated peroxide compound. Illustrative organonitrates or nitric oxide precursors include erythrityl tetranitrate(1,2,3,4-butanetetrol tetranitrate), isosorbide dinitrate, nitroglycerin, pentaerythritol tetranitrate, isosorbide mononitrate and nicorandil[N-[2-(nitroxy)ethyl]-3-pyridinecarboxamide]. An organonitrate compound is also used in a vasodilating effective amount. Methods for measuring vasodilation using an organonitrate are well known as are vasodilating amounts for internal use by oral or buccal administration, as such compounds are commercially available and approved for such uses by governmental bodies of many countries including the US FDA. Determination of a topically applied vasodilating effective amount can therefore be readily determined by a skilled worker.

### Dosage Forms

The present invention also contemplates a pharmaceutical composition suitable for topical administration to human genitalia. A contemplated composition contains an effective sexual arousal-inducing amount of a peroxide compound such as carbamide peroxide or benzoyl peroxide dissolved or dispersed in a pharmaceutically acceptable topical vehicle to provide a composition suitable for topical administration.

Also included in the scope of the invention is the combination of an effective sexual arousal-inducing amount of a peroxide compound such as carbamide peroxide alone or in combination with a vasodilating effective amount of one or more vasodilating agents discussed hereinbefore.

Dosage forms include various pharmaceutically acceptable topical vehicles also referred to in the art as carriers such as gels, creams, ointments. These compositions can, and preferably do, contain substantial amounts of water; i.e., about 50 to about 90 percent by weight,. and more preferably about 60 to about 80 weight percent, or can be substantially free of water, as in the case of an ointment. Aqueous creams and gels are preferred. A cream is an emulsion that preferably has water as the external phase (an oil-in-water emulsion) and has a semi-solid viscosity that resembles the viscosity of mayonnaise. A gel is also a semi-solid, that is somewhat thicker or more viscous than mayonnaise. Such a composition exhibits non-Newtonian flow characteristics that can be described as thixotropic; i.e., the flow is characterized by 1) a yield point, 2) pseudoplastic behavior, 3) a reduction in viscosity on continued shearing, visible over a finite time, and 4) a tendency to rebuild viscosity and/or yield point on standing. Aqueous compositions are illustrated hereinafter as substantially non-aqueous ointments such as those based on petroleum jelly (petrolatum) are more easily prepared.

Other dosage forms for topical and local applications can include but are not limited to, sprays, aerosols, aerosol foams, powders, solutions, suspensions, and emulsions. A formulation can contain all necessary and appropriate pharmaceutical ingredients such as acceptable solvents including alcohols, polyols, esters, and ethers and the like, as well as synthetic, semi-synthetic and natural polymers that act as thickening agents.

A polymeric thickener is present in a thickening effective amount. Such amounts differ with the polymeric agent and composition, but are readily determined by a formulator of ordinary skill. Illustrative polymeric thickeners that are particularly useful in an aqueous composition include semi-synthetic polymers such as the starch derivatives Zeina B862 hydroxypropyl starch phosphate, Zeina B860 hydroxypropyl starch, a synthetic polymer such as the polyacrylic acids discussed hereinafter and the natural polymers such as the polysaccharide gums that are discussed below.

A polysaccharide gum is another useful thickener that can be present in a contemplated composition. Suitable representative gums are those in the galactomannan gum category. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→6). Single membered α-D-manopyranosyl units, linked (1→6) with the main chain, are present as side branches. Galactomannan gums include guar gum, which is the pulverized endosperm of the seed of either of two leguminous plants (cyamposis tetragonalobus and psoraloids) and locust bean gum, which is found in the endosperm of the seeds of the carob tree (ceratonia siliqua). Locust bean gum is preferred for the present invention.

Other suitable representative gums include agar gum, carrageenan gum, ghatti gum, karaya gum, rhamsan gum and xanthan gum. A composition of the present invention can contain a mixture of various gums, or mixture of gums and acidic polymers.

Gums, and galactomannan gums in particular, are well-known materials. See for instance, Industrial Gums: Polysaccharides & Their Derivatives, Whistler R. L. and BeMiller J. N. (eds.), 3rd Ed. Academic Press (1992) and Davidson R. L., Handbook of Water-Soluble Gums & Resins, McGraw-Hill, Inc., New York (1980). Most gums are commercially available in various forms, commonly a powder, and ready for use in foods and topical compositions. For example, locust bean gum in powdered form is available from Tic Gums Inc. (Belcam, Md.).

A polysaccharide gum, when used, is present at about 0.5 percent to about 5 percent, based on the total weight of the composition, with the preferred amount being about 0.5 percent to about 2 percent.

An alternative or addition to the polysaccharide gum is a polyacrylic acid polymer. A common variety of polyacrylic acid polymer is known generically as “carbomer”. Carbomer polymers are polyacrylic acid polymers lightly cross-linked with polyalkenyl polyether. There materials are commercially available from the B. F. Goodrich Company (Akron, Ohio) under the designation “CARBOPOL®” . A particularly preferred variety of carbomer are those designated as “CARBOPOL 940” and “CARBOPOL 934”.

Other polyacrylic acid polymers suitable for use in practicing this invention are those commercially available under the designations “Pemulen®” (B. F. Goodrich Company) and “POLYCARBOPHIL®” (A. H. Robbins, Richmond, Va.). The Pemulen® polymers are copolymers of C₁₀ to C₃₀ alkyl acrylates and one or more monomers of acrylic acid, methacrylic acid or one of their simple esters cross-linked with an allyl ether of sucrose or an allyl ether of pentaerythritol. POLYCARBOPHIL® is a polyacrylic acid cross-linked with divinyl glycol. POLYCARBOPHIL® is used in the vaginal moisturizer disclosed in U.S. Pat. No. 5,474,768.

A non-aqueous topical vehicle is also contemplated, as noted before. Here, the vehicle and the resulting composition contain small amounts of water such as less than about 5 percent by weight and preferable less than about 2 percent by weight, and most preferably less than about 1 weight percent.

Illustrative vehicles are comprised of hydrophobic materials such as petrolatum or C₈-C₂₂ fatty acid esters of C₁-C₆ alcohols. Illustrative materials include methyl palmitate, hexyl laurate, butyl stearate, isopropyl eicosanoate(arachidate), isopropyl behenate and the like. These materials are often used as emollients in the cosmetics industry and are well known in that art. Polyethylene glycols, polypropylene glycols and polyethylene-polypropylene block copolymers, known in the art as poloxamers, that are solid and semi-solid at room temperature are also useful in preparing non-aqueous topical vehicles.

Another component often present in a contemplated composition is a penetration enhancer. Such penetration enhancer compounds are described to a greater extent in U.S. Pat. No. 6,046,244 to Buyuktimkin et al. A contemplated penetration enhancer is an alkyl-2-(N,N-disubstituted amino)alkanoate, an (N,N-disubstituted amino)alkanol alkanoate, or a mixture of the two. For convenient reference, alkyl-2-(N,N-disubstituted amino)alkanoates and (N,N-disubstituted amino)alkanol alkanoates can be grouped together and referred to as alkyl(N,N-disubstituted amino)esters.

An alkyl-2-(N,N-disubstituted amino)alkanoate suitable for the present invention can be represented as follows: 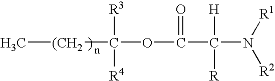
wherein n is an integer having a value of about 4 to about 18; R is a member of the group consisting of hydrogen, C₁-C₇ alkyl, benzyl and phenyl; R¹ and R² are the same or different and are hydrogen or C₁-C₇ alkyl; and R³ and R⁴ are the same or different and are each hydrogen, methyl or ethyl.

Preferred alkyl(N,N-disubstituted amino)alkanoates are C₄-C₁₈ alkyl(N,N-disubstituted amino)acetates and C₄-C₁₈ alkyl(N,N-disubstituted amino)propionates. Exemplary specific alkyl-2-(N,N-disubstituted amino)-alkanoates include dodecyl 2-(N,N dimethylamino)propionate (DDAIP); and dodecyl 2-(N,N-dimethylamino)acetate (DDAA).

Alkyl-2-(N,N-disubstituted amino)alkanoates are known compounds. For example, dodecyl 2-(N,N-dimethylamino)propionate (DDAIP) is available from Steroids, Ltd. (Chicago, Ill.). In addition, alkyl-2-(N,N-disubstituted amino)alkanoates can be synthesized from more readily available compounds as described in U.S. Pat. No. 4,980,378 to Wong et al.

Suitable (N,N-disubstituted amino)alkanol alkanoates for use in a contemplated composition can be represented by the formula: 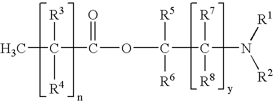
wherein n is an integer having a value of about 5 to about 18; y is an integer having a value of zero to about 5; and R¹, R², R³, R⁴, R⁵, R⁶ and R⁷ are the same or different and are hydrogen, C₁-C₈ alkyl, or C₆-C₈ aryl; and R⁸ is a hydrogen, hydroxyl, C₁-C₈ alkyl or C₆-C₈ aryl.

Preferred (N,N-disubstituted amino)alkanol alkanoates are C₅-C₁₈ carboxylic acid esters. Exemplary specific (N,N-disubstituted amino)alkanol alkanoates include 1-(N,N-dimethylamino)-2-propanol dodecanoate (DAIPD); 1-(N,N-dimethylamino)-2-propanol myristate (DAIPM) and 1-(N,N-dimethylamino)-2-propanol oleate (DAIPO).

The penetration enhancer, when used in a contemplated composition, is present in an amount sufficient to enhance the penetration of at least the peroxide compound and preferably also any vasodilator present in the composition. The specific amount of penetration enhancer varies necessarily according to the desired release rate and the peroxide compound. Generally, this amount of penetration enhancer is about 0.5 percent to about 10 percent, based on the total weight of the composition. Preferably, the penetration enhancer is present at about 5 weight percent of the composition.

Another component that can be present in a contemplated composition is an amphiphilic compound. The term amphiphilic compound as used herein refers to an agent that is both lipophilic and hydrophilic. The C₁-C₈ aliphatic alcohols or alkoxy alcohols, the C₂-C₃₀ aliphatic esters, and their mixtures can serve as amphiphilic compound. Illustrative suitable alcohols are ethanol, n-propanol, isopropanol, propylene glycol, glycerin, diethylene glycol mono ethyl ether, as well as the sugar alcohols such as sorbitol, xylitol, maltitol, lactitol and erythritol, whereas suitable esters are ethyl acetate, butyl acetate, ethyl laurate, methyl propionate and isopropyl myristate. As used herein, the term “aliphatic alcohol” includes polyols such as glycerol, propylene glycol and polyethylene glycols. A mixture of alcohol and ester is preferred, and in particular, a mixture of ethanol and ethyl laurate myristate (a mixture of esters) is particularly preferred.

The concentration of amphiphilic compound utilized necessarily varies according to other factors such as the desired semi-solid consistency and the desired skin penetration promoting effects. One preferred topical composition contains amphiphilic compound in an amount of about 7 percent to about 40 percent by weight based on the total weight of the composition. Where a mixture of aliphatic alcohol and aliphatic ester are employed, the preferred amount of alcohol is about 5 percent to about 15 percent, whereas that of aliphatic ester is about 2 percent to about 15 percent (again based on the total weight of the composition).

An optional, but preferred, component of the present invention is an emulsifier. Although not a critical factor, a suitable emulsifier generally exhibits a hydrophilic-lipophilic balance number greater than 10. Sucrose esters, and specifically sucrose stearate, can serve as emulsifiers for the topical composition of the present invention. Sucrose stearate is a well-known emulsifier available from various commercial sources. Another illustrative emulsifier is a hydrogenated palm oil glyceride [MYVEROL 18-04K} available from Eastman. When an emulsifier is used, the emulsifier such as sucrose stearate is present up to about 2 percent, based on the total weight of the composition, is preferred. The preferred amount of emulsifier can also be expressed as a weight ratio of emulsifier to polysaccharide gum or acrylic acid polymer. A ratio of 1:6 emulsifier:polymer is preferred, and a ratio of 1:4 is more preferred to provide the desired consistency and separation resistance for an aqueous cream (semi-solid).

An acid buffer system is also typically used in aqueous systems. Acid buffer systems serve to maintain or buffer the pH of compositions within a desired range. The term “buffer system” or “buffer” as used herein has reference to a solute agent or agents which, when in a water solution, stabilize such solution against a major change in pH (or hydrogen ion concentration or activity) when acids or bases are added thereto. Solute agent or agents which are thus responsible for a resistance to change in pH from a starting buffered pH value in the range indicated above are well known. Although there are countless suitable buffers, potassium phosphate monohydrate is preferred.

The final pH value of the pharmaceutical composition of the present invention can vary within the physiologically compatible range. Necessarily, the final pH value is not irritating to human skin. Without violating this constraint, the pH value can be selected to adjust consistency when required. With these factors accounted for, the preferred pH value is about 3.0 to about 7.4. The more preferred pH value is about 3.5 to about 6.0.

The remaining component of an aqueous composition is water, which is necessarily purified. The composition contains water at about 50 to about 90 percent, based on the total weight of the composition. The specific amount of water present is usually not critical, however, being adjustable to obtain the desired consistency and/or concentration of the other components.

Additionally, known transdermal penetration enhancers can also be added, if desired. Illustrative are dimethyl sulfoxide (DMSO), dimethyl acetamide (DMA), 2-pyrrolidone, N,N-diethyl-m-toluamide (DEET), 1-dodecylazacycloheptane-2-one (Azone®), N,N-dimethylformamide, N-methyl-2-pyrrolidone, calcium thioglycolate, oxazolidinone, dioxolane derivatives, laurocapram derivatives, and macrocyclic enhancers such as macrocyclic ketones.

A contemplated composition can further include one or more complex forming and stability enhancing agents, antimicrobials, suspending agents, coloring agents, fragrances and other generally regarded as safe (GRAS) ingredients.

A preferred cream, gel or ointment composition can also be applied as it is from a tube or jar, or the composition can filled in an appropriate container or delivery device preferably composed of a tip, a barrel, and a piston to apply to the tip of penis or to the urethral or vaginal opening. It is apparent to those skilled in the art that only typical embodiments have been described by way of exemplification and that there are various modifications, improvements, alterations, and changes that fall in the scope of the present invention to be covered by the claims appended hereto.

### Preperation of Formulations

For the preparation of formulations, carbamide peroxide with or without vasodilating agent, was weighed into a stainless steel container. After addition of polyol solvent or their mixtures, the system was mixed using a Heidolph overhead mixer equipped with propeller type blades mounted on to a stainless steel shaft, and stirred under high shear until dissolution. During this period the mixture was kept at a temperature under 15° C. At the end of this time period, if needed, pharmaceutical aids or similar agents, pharmaceutically acceptable thickening agents such as preferably but not limited to, a polymeric compound such as semi-synthetic phosphated or non-phosphated hydroxypropyl starch or other derivatives, propylene glycol alginates, other synthetic compounds such as carbomers, acrylic polymers, copolymers, naturally occurring nitrogen containing polymers such as collagens, chitins, chitosans and derivatives, hyaluronic acid and derivatives, poloxamers, polyvinyl alcohols and derivatives or natural gums such as locust bean gum, xanthan gum, guar gum or other pharmaceutically acceptable compounds at up to about a 30% level, preferably at about a 10% level, and if needed a buffer system preferably at pH 4-8, most preferably at pH 5.5 were successfully incorporated. A typical formulation is as follows:

| Ingredient | % |
|---|---|
| Carbamide Peroxide USP | 6.5 |
| Diethylene glycol | 10 |
| monoethyl ether | |
| (Transcutol) | |
| Hydroxypropyl Starch | 10 |
| phosphate (Zeina B862) | |
| Buffered Sorbitol 70% | 73.5 |
| solution | |

### Volunteer Trial

The application of the formulations was performed by healthy volunteers with the result that the carbamide peroxide formulations with or without alprostadil showed significant erection and positive arousals. The following example illustrates the effect of the present invention.

Three healthy 53-57 years old male volunteers were given the formulation above containing 6.5 weight percent carbamide peroxide. The formulation (0.5 g) was previously placed into 1 mL tuberculin syringes and slowly applied topically onto the tip of penis at the urethral meatus and particularly to the fossa navicularis. Each individual reapplied the same formulation three times with 3 days between applications so that the applications were done on days one, five and nine. The volunteers were informed that the formulations could either contain active drug or placebo.

A similar study was repeated after two weeks using the same formulations containing carbamide peroxide 6.5 percent w/w but with alprostadil 0.4 percent w/w added to the formulations. At the end of each use of medication, the volunteers were asked to complete a questionnaire, which ask them to describe their responses.

After the evaluation of the responses of all of the volunteers it has been found that the formulation containing only carbamide peroxide as the active ingredient produced a feeling of significant arousal and. erection after light tactile stimulation (holding the penis firmly in one hand for 30 seconds), however, no erections were observed without light tactile stimulation. After using the carbamide peroxide only formulation, the volunteer subjects claimed that they had a better hardness and excellent partner satisfaction. Volunteers did not experience significant pain, irritation or discomfort in the penis or scrotum. Only sensations of warmth and tingling were noted. The results are summarized in Table 1A-1C, below.

TABLE 1A

| | | Interval | | | |
|---|---|---|---|---|---|
| | | Day 1 | | | |
| | | Time to | | | |
| | | Arousal | Arousal | Local Side | |
| Volunteer | Trial | Feeling | Feeling | Effect | Erection |
| #/Age | # | (min)* | Score** | (intensity)*** | Quality**** |
| 1/53 | 1 | 10 | 4 | 1 | 5 |
| | 2 | 11 | 5 | 1 | 5 |
| 2/56 | 1 | 17 | 5 | 1 | 5 |
| | 2 | 14 | 4 | 1 | 5 |
| 3/57 | 1 | 13 | 5 | 1 | 5 |
| | 2 | 16 | 5 | 2 | 5 |
| 4/53 | 1 | 13 | 4 | 1 | 5 |
| | 2 | 12 | 5 | 1 | 5 |

(Notes follow Table 1C)

TABLE 1B

| | | Interval | | | |
|---|---|---|---|---|---|
| | | Day 2 | | | |
| | | Time to | | | |
| | | Arousal | Arousal | Local Side | |
| Volunteer | Trial | Feeling | Feeling | Effect | Erection |
| #/Age | # | (min)* | Score** | (intensity)*** | Quality**** |
| 1/53 | 1 | 15 | 5 | None | 5 |
| | 2 | 14 | 4 | None | 5 |
| 2/56 | 1 | 14 | 5 | 1 | 5 |
| | 2 | 16 | 3 | 1 | 4 |
| 3/57 | 1 | 12 | 4 | None | 5 |
| | 2 | 13 | 5 | 1 | 5 |
| 4/53 | 1 | 10 | 5 | None | 5 |
| | 2 | 9 | 5 | 1 | 5 |

(Notes follow Table 1C)

TABLE 1C

| | | Interval | | | |
|---|---|---|---|---|---|
| | | Day 3 | | | |
| | | Time to | | | |
| | | Arousal | Arousal | Local Side | |
| Volunteer | Trial | Feeling | Feeling | Effect | Erection |
| #/Age | # | (min)* | Score** | (intensity)*** | Quality**** |
| 1/53 | 1 | 14 | 4 | 1 | 5 |
| | 2 | 17 | 5 | None | 5 |
| 2/56 | 1 | 10 | 4 | None | 4 |
| | 2 | 9 | 5 | None | 5 |
| 3/57 | 1 | 11 | 5 | None | 5 |
| | 2 | 15 | 4 | 1 | 5 |
| 4/53 | 1 | 10 | 5 | None | 4 |
| | 2 | 14 | 5 | 1 | 5 |
| *Self-reported time to onset of sexual arousal feeling after the application of the medication. | | | | | |
| **Self-reported arousal feeling score according to a rating scale. Score scale ranges from 0 = no arousal feeling, 1 = slight arousal, 2 = somewhat aroused, 3 = moderately aroused, 4 = very aroused, 5 = extremely aroused | | | | | |
| ***Intensity Scale: 0 = (None) no side effects experienced, 1 = very slight feeling (Warmth and Tingling), 2 = distinctive feeling but not uncomfortable (Warmth and Tingling), 3 = mild feeling but not sufficient to impair activity, 4 = moderate feeling, uncomfortable but activity still possible, 5 = severe feeling sufficient to impair activity. | | | | | |
| ****Intensity of Erection. Scale ranges from 0 to 5. 0 = no erection, 1 = some tumescence but erection not sufficient for intercourse, 2 = moderate tumescence but erection not sufficient for intercourse, 3 = erection sufficient for intercourse and is about the same as the subjects usual erection, 4 = erection sufficient for intercourse and is better | | | | | |
| # than the subjects usual erection, 5 = erection sufficient for intercourse and much better than the subjects usual erection. | | | | | |

As a comparison, formulations containing both of the carbamide peroxide and alprostadil induced a good erection, some burning sensation and sometimes some slight pain and feeling of fullness at the scrotal area. The volunteers also had feeling that they might better tolerate alprostadil/carbamide peroxide combination formulation if a lower dose were given.

### EXAMPLE 2

### Topical Compositions A-F

Composition A is prepared as follows, with compositions B-F being similarly prepared. Part A is formed by dissolving the peroxide compound and prostaglandin E₁ (Alprostadil USP) in 5 parts of ethyl alcohol. Next, 5 parts dodecyl 2-(N,N-dimethylamino)propionate are mixed into the alcohol-peroxide compound-prostaglandin E₁ solution, optionally followed by 5 parts ethyl laurate.

Part B is prepared starting from a pH 5.5 water/buffer solution. The water/buffer solution is prepared by adding sufficient potassium phosphate monohydrate to purified water to create a 0.1 M solution. The pH of the water/buffer solution is adjusted to 5.5 with a strong base solution (1 N sodium hydroxide) and a strong acid (1 N phosphoric acid). Ethyl laurate (0.5 parts) is added to a portion of the buffer solution. Next, the locust bean gum (in powder form) or polycarbophil is dispersed in the buffer solution with the emulsifier and homogenized using a homogenizer. Parts A and B are nixed in the homogenized nd the remaining required water is added.

The resulting compositions are spreadable, semi-solid creams, suitable for application to the genitalia without the need for supporting devices such as patches and adhesive strips. The compositions are both homogenous in appearance.

| | Composition and Amount | | | | | |
|---|---|---|---|---|---|---|
| | (% w/w of Total) | | | | | |
| Ingredient | A | B | C | D | E | F |
| | Part A | | | | | |
| Benzoyl Peroxide | — | — | — | 5 | 10 | 15 |
| Carbamide Peroxide | 5 | 7 | 10 | — | — | — |
| Ethanol | 5 | 5 | 5 | 5 | 5 | 10 |
| Ethyl Laurate | 5 | — | 5 | — | — | — |
| DDAIP | — | 5 | — | 5 | 5 | 5 |
| Prostaglandin E₁ | 0.1 | — | 0.4 | 0.5 | — | — |
| Nitroglycerine | — | 2 | — | — | — | 1 |
| | Part B | | | | | |
| Locust Bean Gum | 3 | 3 | 3 | 3 | — | — |
| (Prehydrated) | | | | | | |
| Polycarbophil | — | — | — | — | 2 | 2 |
| Sucrose Stearate | 0.5 | 0.5 | 0.5 | 0.5 | | |
| a hydrogenated palm | — | — | — | — | 1 | 1 |
| oil glyceride | | | | | | |
| [MYVEROL 18-04K] | | | | | | |
| Water/Buffer (pH 5.5) | qs | qs | qs | qs | qs | qs |

Each of the patents and articles cited herein is hereby incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. A composition for the treatment or prevention of sexual dysfunction comprising an effective sexual arousal-inducing amount of a peroxide compound dissolved or dispersed in a topical vehicle.

2. The composition according to claim 1 that is a semi-solid at room temperature in the form of a gel, cream or ointment.

3. The composition according to claim 1 wherein said topical vehicle is aqueous.

4. The composition according to claim 1 wherein said topical vehicle is non-aqueous.

5. A composition for the treatment or prevention of sexual dysfunction comprising an effective sexual arousal-inducing amount of a peroxide compound dissolved or dispersed in an aqueous topical vehicle that is a cream or gel.

6. The composition according to claim 5 wherein said peroxide compound is benzoyl peroxide, carbamide peroxide or a mixture thereof.

7. The composition according to claim 5 that contains about 50 to about 90 percent by weight water.

8. The composition according to claim 5 that contains an effective amount of a polymeric thickener.

9. A composition for the treatment or prevention of sexual dysfunction comprising an effective sexual arousal-inducing amount of a peroxide compound dissolved or dispersed in a topical vehicle that is a cream or gel, wherein said peroxide compound is benzoyl peroxide, carbamide peroxide or a mixture thereof, and said composition contains about 50 to about 90 percent by weight water and an effective amount of a polymeric thickener.

10. The composition according to claim 9 wherein said composition further includes an effective amount of a vasodilating agent.

11. The composition according to claim 10 wherein said vasodilating agent is prostaglandin E₁, an organonitrate or phentolamine.

12. The composition according to claim 9 wherein said composition further includes an effective amount of a penetrating agent.

13. The composition according to claim 12 wherein said penetrating agent is an alkyl-2-(N,N-disubstituted amino)alkanoate, an (N,N-disubstituted amino)alkanol alkanoate, or a mixture of the two.

14. A composition for the treatment or prevention of sexual dysfunction comprising an effective sexual arousal-inducing amount of a peroxide compound dissolved or dispersed in a non-aqueous topical vehicle that is an ointment.

15. The composition according to claim 14 wherein said peroxide compound is benzoyl peroxide, carbamide peroxide or a mixture thereof.

16. The composition according to claim 14 wherein said composition further includes an effective amount of a vasodilating agent.

17. The composition according to claim 16 wherein said vasodilating agent is prostaglandin E₁, an organonitrate or phentolamine.

18. The composition according to claim 14 wherein said composition further includes an effective amount of a penetrating agent.

19. The composition according to claim 18 wherein said penetrating agent is an alkyl-2-(N,N-disubstituted amino)alkanoate, an (N,N-disubstituted amino)alkanol alkanoate, or a mixture of the two.

20. A method for treatment or prevention of sexual dysfunction that comprises topically applying a composition that contains a sexual arousal-inducing effective amount of a pharmaceutically acceptable peroxide dissolved or dispersed in a topical vehicle to the genitals of a human in need thereof.

21. The method according to claim 20 wherein said topical composition is a semi-solid at room temperature in the form of a gel, cream or ointment.

22. The method according to claim 20 wherein said peroxide compound is benzoyl peroxide, carbamide peroxide or a mixture thereof.

23. The method according to claim 20 wherein said composition is applied about fifteen minutes to about one hour prior to the time at which sexual arousal is desired.

24. The method according to claim 20 wherein said composition further includes an effective amount of a polymeric thickener.

25. The method according to claim 20 wherein said composition further includes an effective amount of a penetrating agent.

26. The method according to claim 20 wherein said composition further includes an effective amount of a vasodilating agent.

27. The method according to claim 20 wherein said topical vehicle is aqueous.

28. The method according to claim 20 wherein said topical vehicle is non-aqueous.
